(19) United States
(12) Patent Application Publication
(10) Pub. No.: US 2011/0033461 A1
(43) Pub. Date: Feb. 10, 2011

(54) Combination Therapy for the Treatment of Cancer

(76) Inventors: Vladimir Ratushny, Woodbury, NY (US); Erica Golemis, Oreland, PA (US); Igor Astsaturov, Philadelphia, PA (US); Iiya G. Serebriiskii, Rockedge, PA (US); Louis M. Weiner, Washington, DC (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/922,310

(22) PCT Filed: Mar. 12, 2009
(86) PCT No.: PCT/US09/36976

§ 371 Date: Oct. 14, 2010

Related U.S. Application Data
(60) Provisional application No. 61/036,027, filed on Mar. 12, 2008.

(51) Int. Cl. A61K 39/395; A61K 31/713; A61K 31/55; A61K 31/496; A61K 31/517; A61K 31/5377; A61K 38/47; A61K 38/16; A61K 31/675; A61K 33/24; A61K 31/573; A61K 38/46; A61K 31/706; A61K 31/568; A61K 31/7072; A61K 31/565; A61K 31/57; A61K 38/08; A61P 35/00; A61P 35/04; A61K 38/09

(52) U.S. Cl. 424/133.1; 424/142.1; 514/44 A; 514/215; 514/252.19; 514/254.06; 514/266.4; 514/233.8; 424/94.61; 514/19.3; 514/19.8; 514/90; 424/649; 514/177; 424/94.6; 514/43; 514/178; 514/50; 514/171; 514/9.7; 514/19.4; 514/19.5; 514/10.4; 514/10.3

(57) ABSTRACT

Compositions which act synergistically to inhibit the growth of cancer cells and methods of use thereof are disclosed.

This application claims priority to U.S. Provisional Application No. 61/036,027, filed Mar. 12, 2008.

### FIELD OF THE INVENTION

The present invention relates to the fields of drug discovery and oncology. More specifically, the invention provides a combination of agents that act synergistically to inhibit the growth of cancer cells and methods of use thereof for the treatment of cancer. Pharmaceutical compositions comprising the agents of the invention for the treatment of malignancy and other disorders associated with aberrant cellular proliferation are also disclosed.

### BACKGROUND OF THE INVENTION

Several publications and patent documents are cited throughout the specification in order to describe the state of the art to which this invention pertains. Each of these citations is incorporated herein by reference as though set forth in full.

The National Cancer Institute has estimated that in the United States alone, 1 in 3 people will be struck with cancer during their lifetime. Moreover approximately 50% to 60% of people contracting cancer will eventually succumb to the disease. The widespread occurrence of this disease underscores the need for improved anticancer regimens for the treatment of malignancy.

In the past several years, Aurora-A kinase (AurA) has attracted increasing attention because it is overexpressed in a high percentage of tumors arising in breast, colon, ovary, and other tissues, and because it has been shown to function as an oncogene when exogenously expressed in various cell line models. Auroroa-A kinase overexpression, whether in naturally occurring tumors or following deliberate overexpression, is associated with increased numbers of centrosomes and multipolar spindles, which arise as a consequence of failed cytokinesis. As the overexpressed Aurora-A kinase is not limited to expression in G2 and M phases at the centrosome, but is also detected throughout the cytoplasm in cells in different cell cycle compartments, it is not clear at present whether the transforming activity of Aurora-A kinase arises from hyperactivation of normal Aurora-A kinase substrates, or through anomalous targeting by Aurora-A kinase of additional substrates. Unexpectedly, even overexpression of a kinase-inactive form of Aurora-A kinase can induce supernumerary centrosomes (although it cannot transform cells), supporting the idea that the protein has at least two different functions in regulating centrosome numbers: a kinase function, and a scaffolding function for other proteins.

Based on these various properties, Aurora-A kinase is now being actively exploited as a target for development of new anti-cancer agents (reviewed in Andrews, P. D. Aurora kinases: shining lights on the therapeutic horizon? Oncogene 2005; 24:5005-15.). The PHA-680632 Aurora-A kinase-inhibiting compound developed by Nerviano-MS, currently in clinical trials, has been described in the studies described in Soncini, C. et al. (PHA-680632, a novel Aurora kinase inhibitor with potent anti-tumoral activity. Clin Cancer Res (2006) 12: 4080-4089). This compound targets Aurora-A kinase preferentially, but also targets a related protein, Aurora-B kinase, which has also been implicated as an oncogene in some cancers. Other agents target Aurora-A kinase exclusively, including C1368 (Sigma), and MLN8054, developed by Millenium Pharmaceuticals (currently in Phase I trials). Another Aurora kinase inhibitor, PHA-739358 (Nerviano) also shows promise for use in anti-cancer regimens (Modugno et al. (2007) Cancer Res. 67:7987). VX-680/MK0457 (Vertex) also targets Aurora-A and Aurora-B kinases and is currently being evaluated in clinical trials. AKI-001 (Genentech, Inc.) likewise shows inhibitory action against both Aurora-A kinase and Aurora-B kinase (Rawson et al. J Med Chem. (2008) 14:4465-75). Schellens et al. have reported on Phase I and pharmacological studies of AZD1152 (Astra Zeneca), an Aurora-B kinase inhibitor (Journal of Clinical Oncology, (2006) 24: No 18S (June 20 Supplement), 2006: 3008).

Despite millions of dollars being spent each year in efforts to identify effective anti-cancer agents and treatment regimens, cancer has yet to be eradicated and effective treatment regimens that are not overly toxic to the patient are still limited in number. It is clear that a need exists for improved anti-neoplastic agents, and for methods of use thereof for the treatment of malignant disease.

### SUMMARY OF THE INVENTION

The present invention provides effective therapeutic methods for modulating tumor growth or metastasis wherein a combination of agents is employed. The methods of the present invention provide advantages such as greater overall efficacy, for example, in achieving synergy or avoiding antagonism, and allow, where desired, a reduction in the amount of one or more of the individual agents employed with a concomitant reduction in side effects. Further, where the tumor to be treated is not optimally responsive to a given anticancer agent, use of the present combination therapy methods can nonetheless provide effective treatment.

In particular, the present invention provides a method for modulating tumor growth or metastasis in a subject, especially a human, in need thereof, comprising sequential or simultaneous administration of at least one Aurora kinase inhibitor and at least one EGFR inhibitor in amounts effective therefore. Preferred Aurora kinase inhibitors include, without limitation, VX-680, AKI-001, PHA-680632, PHA-739358, MLN8054, MLN8237 and agents which down modulate expression thereof, e.g., siRNA or antisense which hybridize to Aurora kinase encoding nucleic acids. Preferred EGFR inhibitors include, for example, erlotinib, cetuximab, gefinitib and panitumumab. Where simultaneous administration of the Aurora kinase inhibitor and at least one EGFR inhibitor is desired, the present invention also provides pharmaceutical compositions comprising these agents in a sub-therapeutic dose for the individual agent, the agents being effective in combination, providing reduced side effects while maintaining efficacy. Alternatively, each agent can be provided at higher doses for the individual agent. Alternatively, where simultaneous or sequential administration of the Aurora kinase inhibitor and EGFR inhibitor is contemplated, the present invention further provides a first pharmaceutical composition comprising at least one Aurora kinase inhibitor and a second pharmaceutical composition comprising at least one EGFR inhibitor together in a package or kit. Preferably, the Aurora kinase inhibitor is selected from the group comprising VX-680, PHA-68032, PHA-739358 and MLN8054, MLN 8237 and the EGFR inhibitor is selected from the group comprising erlotinib, cetuximab, gefitinib, panitumumab, or other related agents. In yet another aspect, the methods and compositions described above can further include at least one anti-cancer, anti-angiogenic, or anti-proliferative agent for the treatment and management of cancer and other disorders characterized by aberrant cellular proliferation.

### BRIEF DESCRIPTION OF THE DRAWINGS

FIG. 1 is a table showing that 3 out of 4 siRNA targeting Aurora kinase A sensitize HCT116 cells to erlotinib.

FIG. 2 is a series of histograms that show that the EGFR inhibitor erlotinib does not appear to affect the HCT116 cell cycle.

FIGS. 3A-3D are a series of graphs showing synergy between the Sigma Aurora kinase inhibitor C1368 and erlotinib in HCT116 cells.

FIGS. 4A-4D are a series of graph showings synergy between the Sigma Aurora kinase inhibitor C1368 and erlotinib in a second Ras-mutated colorectal cancer cell line, DLD-1.

FIG. 5 shows a median effect plot, dose-effect curve and FaCI plot of the synergistic effect exhibited by a combined administration of C1368 and erlotinib.

FIGS. 6A-6D show the synergistic effect of cetuximab and an Aurora kinase inhibitor (1:109 ratio) in HCT116 cells.

FIG. 7 shows a median effect plot, dose-effect curve, and FaCI plot of the strong synergistic effect exhibited by a combined administration of C1368 and erlotinib.

### DETAILED DESCRIPTION OF THE INVENTION

In accordance with the present invention, we have determined that the combination of chemical inhibition of Aurora kinases with inhibition of EGFR results in the synergistic inhibition of cancer cell growth. In a preferred embodiment, the Aurora kinase inhibitor is selected from the group consisting of siRNA which down modulate expression of Aurora kinase, bioavailable small molecule inhibitors of Aurora kinases (both A and B), e.g., VX-680 (also known as MK0457; Vertex) PHA-680632 and PHA-739758 (Nerviano), AKI-001 (Genentech), MLN8054, MLN8237 (Millenium Pharmaceuticals), C1368 (Sigma) and the EGFR inhibitor is selected from the group consisting of erlotinib, cetuximab, gefitinib, and panitumumab. In vitro, viability-based synergy experiments detected a strong synergy between the two inhibitor agents.

### DEFINITIONS

The phrase “Aurora kinase inhibitor” refers to any agent which functions to inhibit or down regulate Aurora-A kinase and/or Aurora-B kinase. Such agents include, without limitation, small molecules, chemical compounds and nucleic acid molecules which function to down regulate expression of target genes. Exemplary agents include VX-680 (also known as MK0457; Vertex, AKI-001 (Genentech) PHA-680632, PHA-739358 (Nerviano), C1368 (Sigma), MLN8054 and MLN8237 (Millenium Pharmaceuticals), and siRNA which hybridize selectively to Aurora kinase encoding mRNA and down regulate expression of the aurora kinase protein product. Exemplary siRNAs that target Aurora kinase have the following sequence:

Hs_AURKA_1
TCCCAGCGCATTCCTTTGCAA
and
Hs_STK6_5
CACCTTCGGCATCCTAATATT.

The phrase “EGFR inhibitor” refers to any agent which is effective to impede or inhibit the function of the epidermal growth factor receptor. Such agents include, without limitation, small molecules, chemical compounds and nucleic acid molecules which function to down regulate expression of target genes, such as the EGFR. Exemplary agents include, without limitation, erlotinib (also known as Tarceva®; Genentech), cetuximab (also known as Erbitux®; Bristol Myers Squibb), gefinitib (also known as Iressa®; Astra Zeneca), and panitumumab (also known as Vectibix®; Amgen).

“Anti-cancer or anti-proliferative agents” are compounds that exhibit anticancer activity and/or are detrimental to a cell (e.g., a toxin). In anti-cancer applications, it may be desirable to combine administration of the Aurora-A/Aurora-B kinase inhibitors and EGFR inhibitors described herein with administration of anti-proliferative agents. Suitable agents for this purpose include, but are not limited to: toxins (e.g., saporin, ricin, abrin, ethidium bromide, diptheria toxin, Pseudomonas exotoxin, and others listed above); alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). Anti-angiogenic agents can include VEGF inhibitors, combretastatin and derivatives thereof, bevacizumab (Avastin®), and sorafenib. Additional agents can include monoclonal antibodies targeting additional EGFR family members, e.g. lapjatinib, trastuzumab, ras pathway targeted inhibitors (i.e., Novartis Raf265) and mTOR inhibitors (e.g., temsirolimus).

As used herein, the terms “modulate”, “modulating” or “modulation” refer to changing the rate at which a particular process occurs, inhibiting a particular process, reversing a particular process, and/or preventing the initiation of a particular process. Accordingly, if the particular process is tumor growth or metastasis, the term “modulation” includes, without limitation, decreasing the rate at which tumor growth and/or metastasis occurs; inhibiting tumor growth and/or metastasis; reversing tumor growth and/or metastasis (including tumor shrinkage and/or eradication) and/or preventing tumor growth and/or metastasis.

As used herein, the phrase “effective amount” of a compound or pharmaceutical composition refers to an amount sufficient to modulate tumor growth or metastasis in an animal, especially a human, including without limitation decreasing tumor growth or size or preventing formation of tumor growth in an animal lacking any tumor formation prior to administration, i.e., prophylactic administration.

As used herein, the terms “tumor”, “tumor growth” or “tumor tissue” can be used interchangeably, and refer to an abnormal growth of tissue resulting from uncontrolled progressive multiplication of cells and serving no physiological function. A solid tumor can be malignant, e.g. tending to metastasize and being life threatening, or benign. Examples of solid tumors that can be treated or prevented according to a method of the present invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, gastic cancer, pancreatic cancer, breast cancer, ovarian cancer, prostate cancer, squamous cell carcinoma of the head and neck, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, liver metastases, bile duct carcinoma, choriocarcinoma, seminoma, embryonal carcinoma, thyroid carcinoma such as anaplastic thyroid cancer, Wilms' tumor, cervical cancer, testicular tumor, lung carcinoma such as small cell lung carcinoma and non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Moreover, tumors comprising dysproliferative changes (such as metaplasias and dysplasias) can be treated or prevented with a pharmaceutical composition or method of the present invention in epithelial tissues such as those in the cervix, colon, esophagus, and lung. Thus, the present invention provides for treatment of conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W.B. Saunders Co., Philadelphia, pp. 68 to 79). Hyperplasia is a form of controlled cell proliferation involving an increase in cell number in a tissue or organ, without significant alteration in structure or function. For example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. For a review of such disorders, see Fishman et al., 1985, Medicine, 2d Ed., J. B. Lippincott Co., Philadelphia.

### Pharmaceutical Compositions

As explained above, the present methods can, for example, be carried out using a single pharmaceutical composition comprising both an Aurora kinase inhibitor and EGFR inhibitor (e.g. erlotinib) (when administration is to be simultaneous) or using two or more pharmaceutical compositions separately comprising the Aurora kinase inhibitor and erlotinib (when administration is to be simultaneous or sequential). The phrase “pharmaceutically acceptable” refers to molecular entities and compositions that are physiologically tolerable and preferably do not produce an allergic or similar untoward reaction, such as gastric upset, dizziness and the like, when administered to a human. Notably, the majority of the inhibitors disclosed for use in the present invention are currently being assessed in clinical trial utilizing other protocols. Accordingly, the skilled clinician is readily able to arrive at appropriate dosing and formulations depending on the disease and the condition of the patient to be treated.

Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers, for example to a diluent, adjuvant, excipient, auxilliary agent or vehicle with which an active agent of the present invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin.

A pharmaceutical composition of the present invention can be administered by any suitable route, for example, by injection, by oral, pulmonary, nasal or other forms of administration. In general, pharmaceutical compositions contemplated to be within the scope of the invention, comprise, inter alia, pharmaceutically acceptable diluents, preservatives, solubilizers, emulsifiers, adjuvants and/or carriers. Such compositions can include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington's Pharmaceutical Sciences, 18th Ed. (1990, Mack Publishing Co., Easton, Pa. 18042) pages 1435-1712 which are herein incorporated by reference. A pharmaceutical composition of the present invention can be prepared, for example, in liquid form, or can be in dried powder, such as lyophilized form. Particular methods of administering such compositions are described infra.

### Methods for Modulating Tumor Growth or Metastasis

As explained above, the present invention is directed towards methods for modulating tumor growth and metastasis comprising, the administration of an Aurora-A and/or Aurora-B kinase inhibitor and at least one EGFR inhibitor. The agents of the invention can be administered separately (e.g, formulated and administered separately), or in combination as a pharmaceutical composition of the present invention. Administration can be achieved by any suitable route, such as parenterally, transmucosally, e.g., orally, nasally, or rectally, or transdermally. Preferably, administration is parenteral, e.g., via intravenous injection or oral. Alternative means of administration also include, but are not limited to, intra-arteriole, intramuscular, intradermal, subcutaneous, intraperitoneal, intraventricular, and intracranial administration, or by injection into the tumor(s) being treated or into tissues surrounding the tumor(s).

The Aurora kinase inhibitor and EGFR inhibitor may be employed in any suitable pharmaceutical formulation, as described above, including in a vesicle, such as a liposome [see Langer, Science 249:1527-1533 (1990); Treat et al., in Liposomes in the Therapy of Infectious Disease and Cancer, Lopez-Berestein and Fidler (eds.), Liss: New York, pp. 317-327, see generally, ibid]. Preferably, administration of liposomes containing the agents of the invention is parenteral, e.g., via intravenous injection, but also may include, without limitation, intraarteriole, intramuscular, intradermal, subcutaneous, intraperitoneal, intraventricular, and intracranial administration, or by injection into the tumor(s) being treated or into tissues surrounding the tumor(s).

In yet another embodiment, a pharmaceutical composition of the present invention can be delivered in a controlled release system, such as using an intravenous infusion, an implantable osmotic pump, a transdermal patch, liposomes, or other modes of administration. In a particular embodiment, a pump may be used [see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)]. In another embodiment, polymeric materials can be used [see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)]. In yet another embodiment, a controlled release system can be placed in proximity of the target tissues of the subject, thus requiring only a fraction of the systemic dose [see, e.g., Goodson, in Medical Applications of Controlled Release, supra, vol. 2, pp. 115-138 (1984)]. In particular, a controlled release device can be introduced into a subject in proximity of the site of inappropriate immune activation or a tumor. Other controlled release systems are discussed in the review by Langer [Science 249:1527-1533 (1990)].

The compositions and methods may also include administration of at least one anti-proliferative or anti-cancer agent as described herein above.

The following examples are provided to illustrate certain embodiments of the invention. They are not intended to limit the invention in any way.

### Example I

### Inhibitors of Aurora Kinase and EGFR Act Synergistically to Inhibit the Growth of Cancer Cells

Aurora Kinase, plays an important role in mitotic chromosomal segregation and division and a variety of small inhibitory molecules targeting this enzyme are currently in clinical trials. Erlotinib (Tarceva, Genentech) is a small molecule inhibitor of EGFR tyrosine kinase. It has been FDA approved for the treatment of chemotherapy-resistant non-small cell lung cancer, and as part of a combination therapy for the treatment of pancreatic cancer. No obvious, direct functional connections between the actions of Aurora kinase inhibitors and EGFR inhibitors have ever been reported.

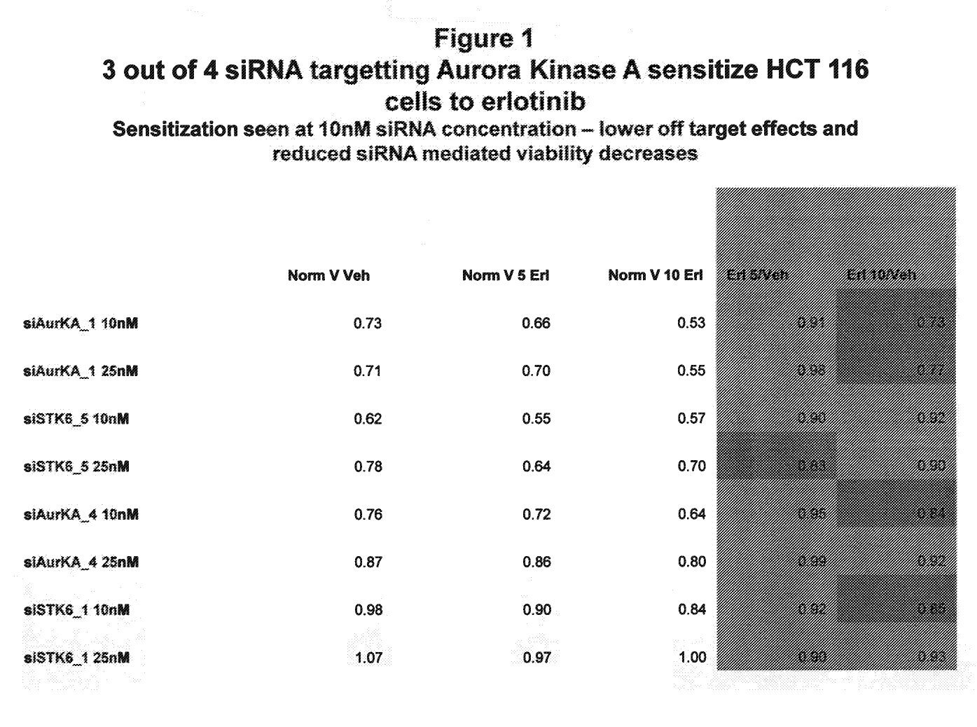

Our first hint of possible synergy between the two agents came as a result of preliminary data from an siRNA chemosensitization screen. As part of our screen, siRNA targeting Aurora-A kinase resulted in sensitization of human colorectal cancer cell line containing a K-Ras mutation, HCT116, to erlotinib. In subsequent deconvolution experiments, three out of four independent, unique siRNA targeting Aurora A kinase yielded erlotinib dose-dependent sensitization of HCT116 cells to erlotinib-induced cell death, although the degree of sensitization was on the weaker end of positive hits in the screen (FIG. 1). However, since kinases are catalytic, we reasoned that an siRNA was less likely to fully ablate Aurora A kinase activity than a small molecule inhibitor. Accordingly, we decided to conduct additional studies using small molecule inhibitors of Aurora kinase.

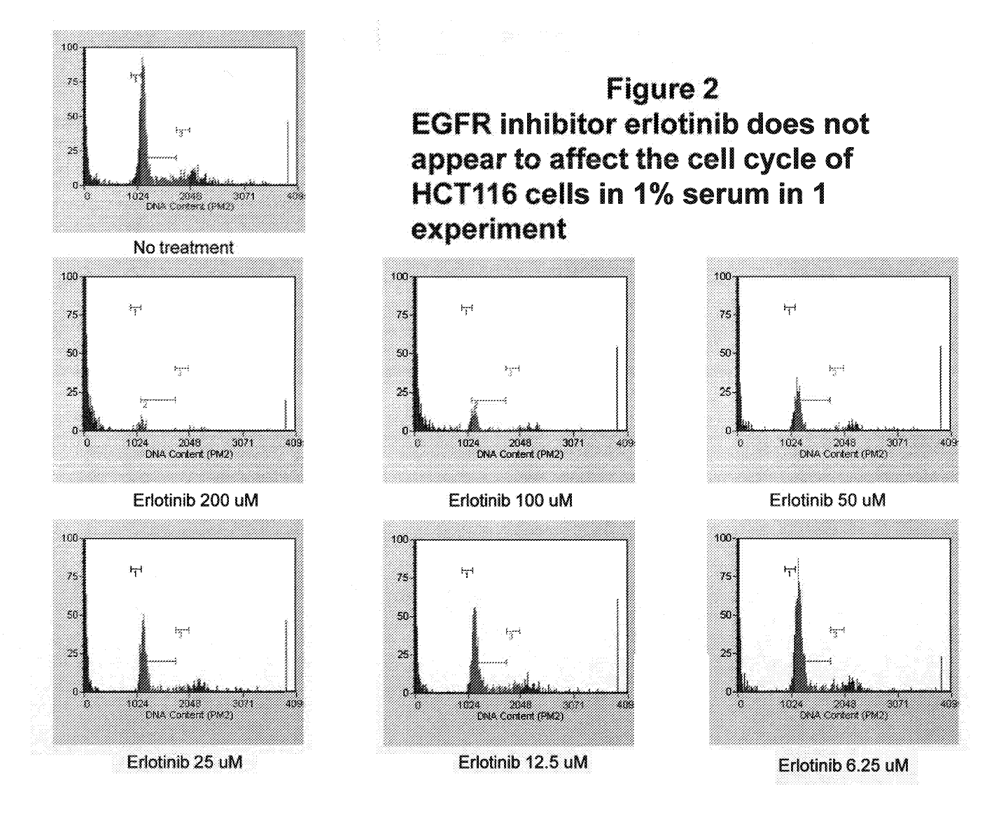

Inhibition of Aurora kinase activity is associated with cell cycle arrest. In contrast, treatment of cells with erlotinib did not noticeably induce cell cycle arrest, although at very high concentrations (>50 μM), there was a reduction in cell number suggesting cell death (FIG. 2). Very different results were seen in analysis of the synergy experiment.

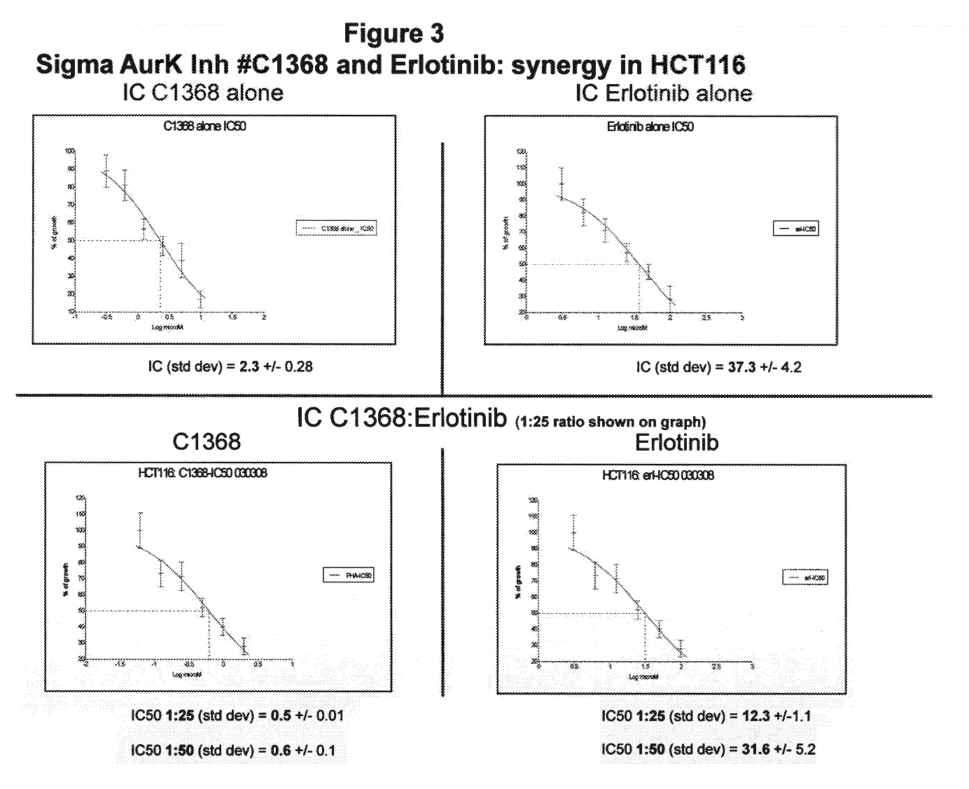

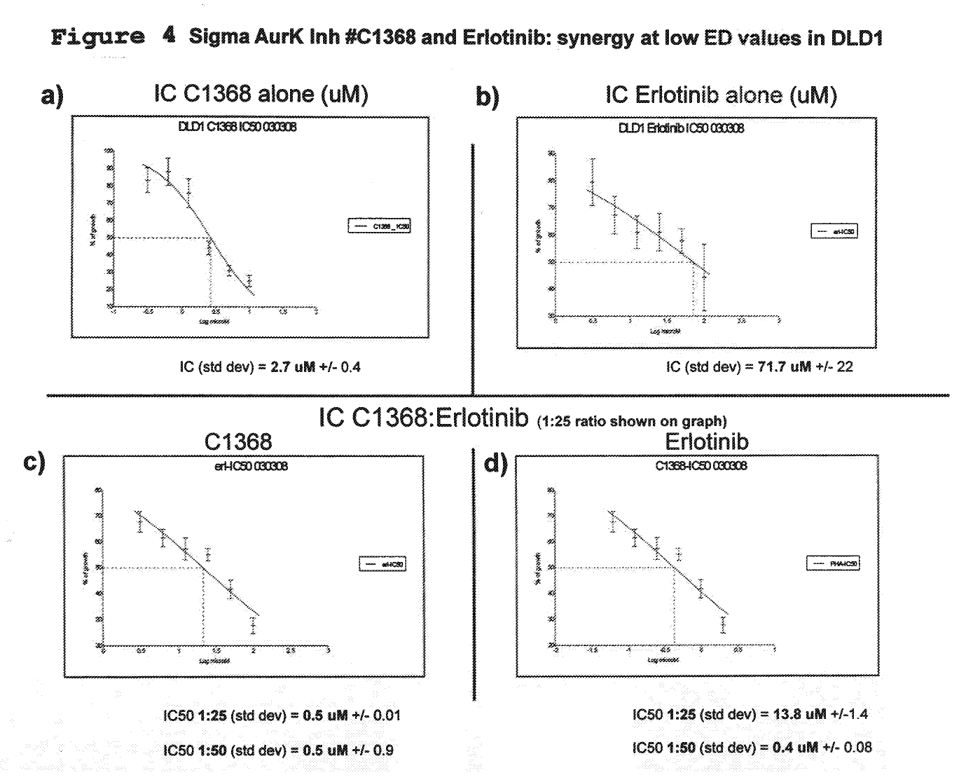

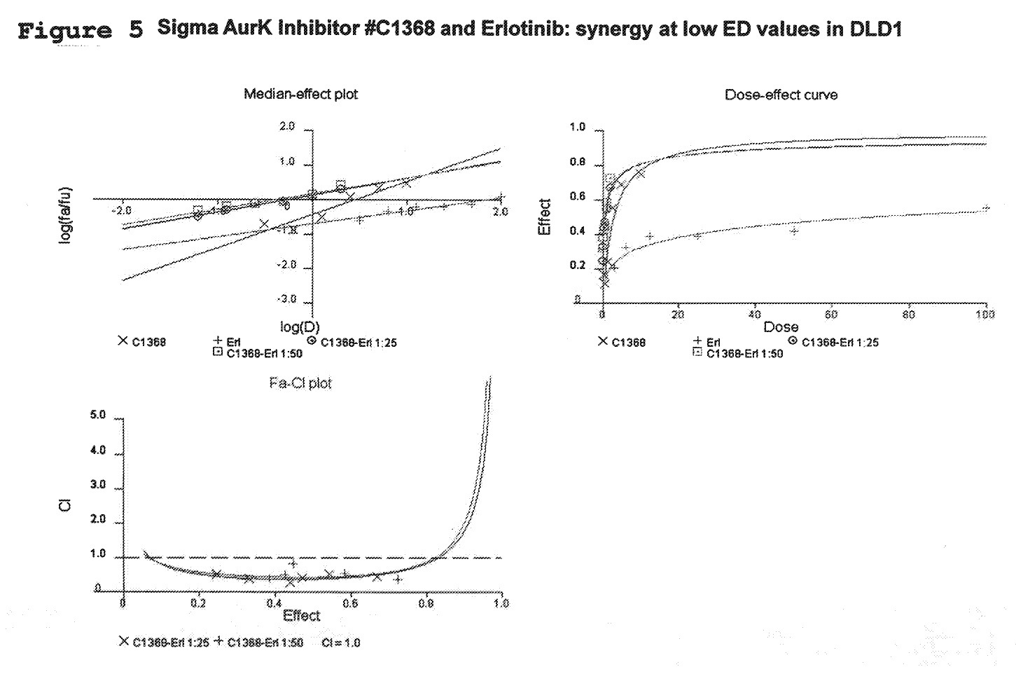

To validate and extend our results, we next tested for synergy with erlotinib using a different kinase inhibitor specific for Aurora Kinase A (Sigma's C1368). When HCT116 cells were treated with a combination (1:25 ratio) of C1368 and erlotinib (1:25 ratio), the 1050 value of erlotinib decreased >3 fold (from 37.3 μM to 12.3 μM) and the 1050 value for C1368 decreased >4 fold (from 2.3 μM to 0.5 μM) as compared to treatment with either drug alone (FIG. 3). Similarly, combination treatment of a second K-Ras-mutated cell line, DLD1, with C1368 and erlotinib resulted in an even greater fold decrease of the 1050 values of the single agents, with IC50 reduced from 71.7 μM to 13.8 μM for erlotinib, and from 2.7 μM to 0.5 μM for C1368 (FIGS. 4 and 5). Synergy is evident for the C1368 and erlotinib combination in both cell lines under both Effective Dose 50 (ED 50) and ED 75, but not at ED 90 (Table 1). ED refers to the percentage of cell killed at different drug doses. The lack of synergy at ED90 is likely due to off-target kinase inhibition, which can occur when C1368 is given at high concentrations.

TABLE 1

| | CI Values at | | |
|---|---|---|---|
| | ED50 | ED75 | ED90 |
| HCT116 cells | | | |
| C1368:Erl1:25 | 0.55777 | 0.82674 | 1.2887 |
| C1368:Erl1:50 | 1.23066 | 1.12096 | 1.05687 |
| DLD1 Cells | | | |
| C1368:Erl1:25 | 0.39 | 0.68 | 1.76 |
| C1368:Erl1:50 | 0.45 | 0.71 | 1.00 |

Data show that C1368 and erlotinib synergize at lower relevant concentrations of the drugs (i.e., ED50 and ED75). Moreover there is a dramatic increase in IC50 values of both drugs when used in combination.

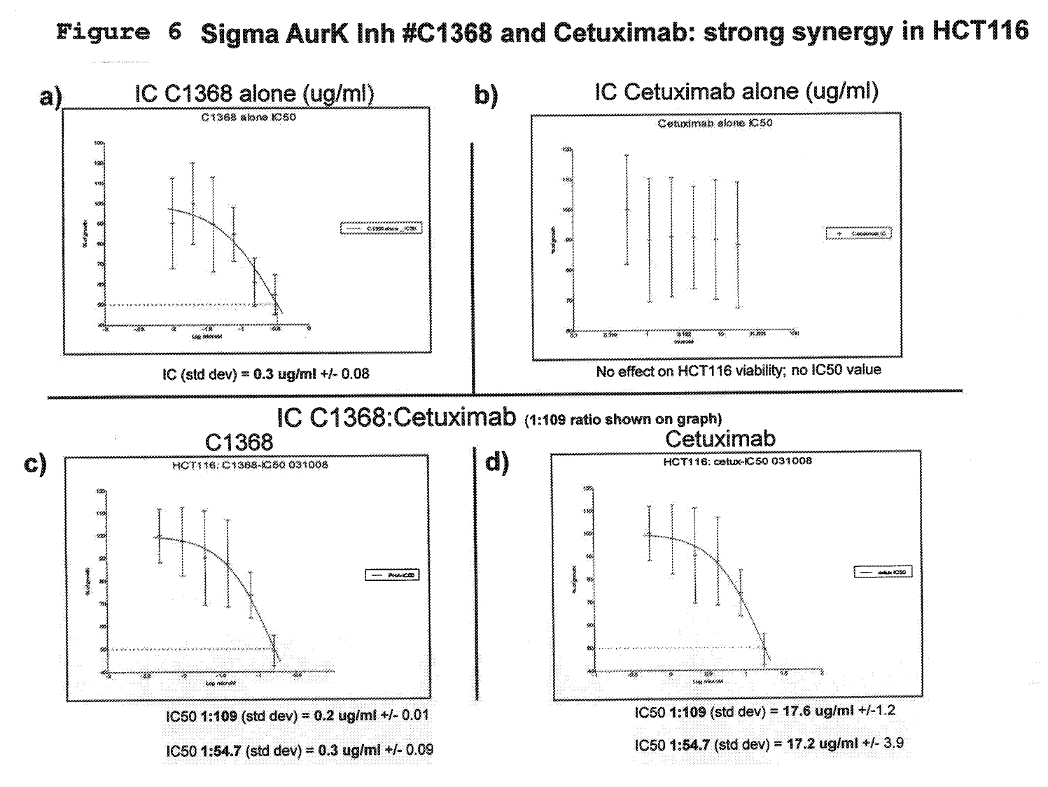

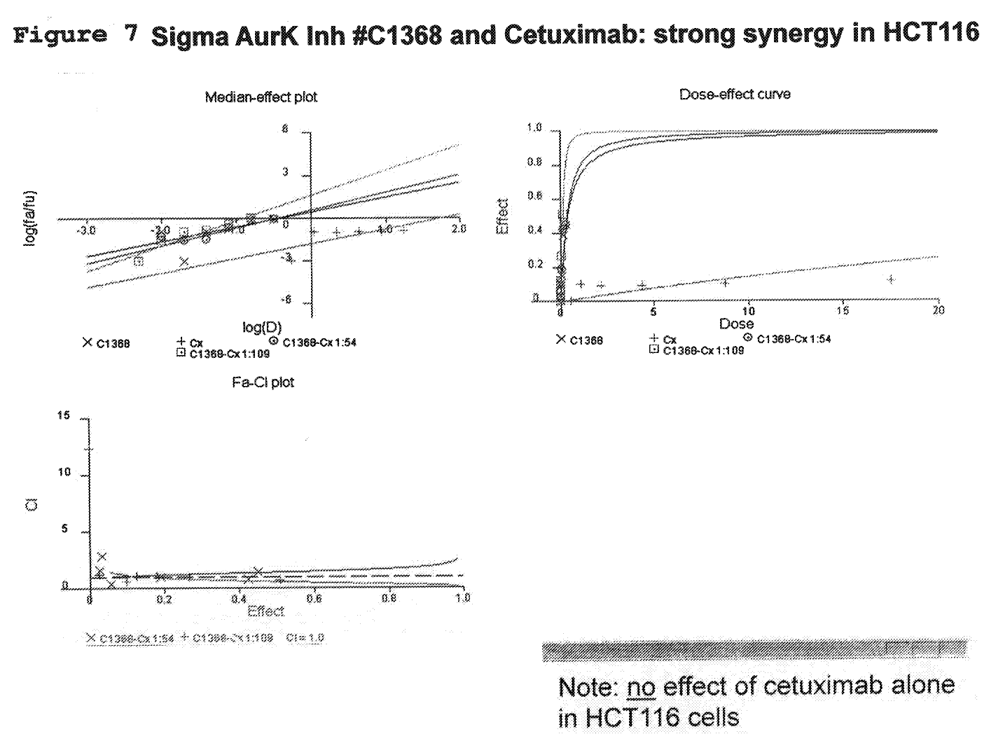

To assess the applicability of our discovery to other FDA approved EGFR inhibitors, we tested the ability of EGFR-targeting antibody, cetuximab, to synergize with Sigma C1368 (FIG. 6). Cetuximab has a completely different mechanism of EGFR inhibition than erlotinib. Cetuximab binds to the cell surface domains of EGFR and causes EGFR internalization and inhibition of ligand mediated signaling. In vivo, cetuximab also induces a combined innate and adaptive immune response to EGFR overexpressing cancer cells, and is known to be much less potent in cultured cells in which these mechanisms do not apply. Cetuximab alone did not cause any significant inhibition of HCT116 cells, and it was impossible to obtain an IC value, (FIG. 6B). Strikingly, cetuximab synergistically killed HCT116 cells when given in combination (1:109 ratio) (1:8.5 ratio in μm) with C1368 (FIGS. 6C and 6D, FIG. 7 and Table 2). The IC50 value of C1368 was reduced from 2.1 μM to 1.0 μM, while an IC50 of cetuximab emerged at 0.1 μM. The CI (avg. 0.43) for C1368 and cetuximab at the 1:109 (8.5:1 ratio in μM) (ratio indicated a strong synergy (Table 2).

TABLE 2

| | CI Values at | | | |
|---|---|---|---|---|
| Drug | ED50 | ED75 | ED90 | CI Avg. |
| C1368-Cx1:109 | 0.58 | 0.41 | 0.30 | 0.43 |

### Example II

### Establishment of Xenograft Models of Human Cancer for Optimizing Methods of Treating Cancer Using the Synergistic Combinations of the Invention

Most cancers of the major organ systems can be excised and cultured in nude mice as xenografts. Additionally, blood born cancers such as leukemias and lymphomas can be established in mice. Such mice provide superior in vivo models for studying the effects of the anti-cancer combinations disclosed herein. The particular cancer types that can be cultured in this way, include without limitation, breast cancer, colon cancer, pancreatic cancer, prostate cancer, ovarian cancer, lung cancer, kidney cancer, stomach cancer, esophageal cancer, and brain cancer. Creating mice comprising such xenografts is well within the purview of the skilled artisan. See for example, “Tumor Models in Cancer Research” (Cancer Drug Discovery and Development) by Beverly A. Teicher (2002) Humana Press, and “Mouse models of Human Cancer” by Eric Holland Cancer Cell (2004) 6:197-8.

Immunocompromised mice are obtained and tumor cells implanted or injected via the tail vein. The cells implanted can include tumor tissue or cells excised from a patient or immortalized cells corresponding to particular cancer types which are commercially available from the ATCC. Once tumors begin to form, the mice can be treated with the synergistic anti-cancer pharmaceutical compositions described herein in order to further characterize dosing, route of administration and timing between subsequent administration of the agents disclosed. Additional combinations of anti-cancer or anti-proliferative agents can also be assessed using such xenograft models and the effects on reduction of tumor burden, tumor cell morphology, tumor invasive properties, angiogenesis, apoptosis, metastasis, morbidty and mortality determined.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1. A method for modulating tumor growth or metastasis in a subject in need thereof, comprising sequential or simultaneous administration of at least one Aurora kinase inhibitor and at least one EGFR inhibitor in amounts effective therefore, said method optionally comprising administration of at least one anti-proliferative agent.

2. The method of claim 1, wherein said Aurora kinase inhibitor is selected from the group consisting of PHA-680632, AKI-001, VX-680, PHA-739358, MLN8054, MLN 8237, C1368 and siRNA which hybridize selectively to aurora kinase.

3. The method of claim 1, wherein said EGFR inhibitor is selected from the group consisting of erlotinib, cetuximab, gefinitib, and panitumumab.

4. The method of claim 1, wherein said Aurora kinase inhibitor is VX-680 and said EGFR inhibitor is erlotinib.

5. The method of claim 1, wherein said Aurora kinase inhibitor is MLN8054 and said EGFR inhibitor is erlotinib.

6. The method of claim 1, wherein said Aurora kinase inhibitor is PHA-680632 and said EGFR inhibitor is erlotinib.

7. The method of claim 1, wherein said Aurora kinase inhibitor is MLN8237 and said EGFR inhibitor is erlotinib.

8. The method of claim 1, wherein said Auroroa kinase inhibitor is PHA-680632 and said EGFR inhibitor is cetuximab.

9. The method of claim 1, wherein said Aurora kinase inhibitor is siRNA which down modulates Aurora kinase expression and said EGFR inhibitor is erlotinib.

10. The method of claim 1 to claim 9 further comprising administration of an effective amount of at least one anti-proliferative agent.

11. The method of claim 10, wherein said at least one anti-proliferative agent is selected from the group consisting of a toxin, saporin, ricin, abrin, ethidium bromide, diptheria toxin, Pseudomonas exotoxin, an alkylating agent, a nitrogen mustards, chlorambucil, cyclophosphamide, isofamide, mechlorethamine, melphalan, uracil mustard; aziridines, thiotepa; a methanesulphonate ester, busulfan; carmustine, lomustine, streptozocin; cisplatin, carboplatin; mitomycin, procarbazine, dacarbazine and altretamine, bleomycin, amsacrine, dactinomycin, daunorubicin, idarubicin, mitoxantrone, doxorubicin, etoposide, teniposide, plicamydin, methotrexate, trimetrexate; fluorouracil, fluorodeoxyuridine, CB3717, azacitidine, cytarabine, floxuridine; mercaptopurine, 6-thioguanine, fludarabine, pentostatin; asparginase, hydroxyurea, vincristine, vinblastine, paclitaxel (Taxol), estrogens; conjugated estrogens; ethinyl estradiol; diethylstilbesterol; chlortrianisen; idenestrol; hydroxyprogesterone caproate, medroxyprogesterone, megestrol; testosterone, testosterone propionate, fluoxymesterone, methyltestosterone, abarelix abiraterone acetate, Degarelix, prednisone, dexamethasone, methylprednisolone, and prednisolone, leuprolide acetate, goserelin acetate, tamoxifen, flutamide, mitotane, and aminoglutethimide, combretastatin and derivatives thereof, bevacizumab (Avastin®), sorafenib, trastuzumab, Raf265 and temsirolimus.

12. A method as claimed in claim 11, wherein said subject has a cancer selected from the group consisting of colorectal cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, gastic cancer, pancreatic cancer, breast cancer, ovarian cancer, prostate cancer, squamous cell carcinoma of the head and neck, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, liver metastases, bile duct carcinoma, choriocarcinoma, seminoma, embryonal carcinoma, thyroid carcinoma such as anaplastic thyroid cancer, Wilms' tumor, cervical cancer, testicular tumor, lung cancer, small cell cancer of the lung, non-small cell cancer of the lung, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

13. A pharmaceutical composition for modulating tumor growth or metastasis in a subject in need thereof, comprising an effective amount of at least one Aurora kinase A inhibitor and at least one EGFR inhibitor, in a pharmaceutically acceptable carrier.

14. A plurality of pharmaceutical compositions for combined administration for modulating tumor growth or metastasis in a subject in need thereof, comprising at least one Aurora kinase A inhibitor and at least one EGFR inhibitor, in amounts effective therefore in separate pharmaceutically acceptable carriers, said composition optionally comprising an effective amount of at least one antiproliferative agent.

15. The pharmaceutical composition of claim 14, wherein said Aurora kinase inhibitor is selected from the group consisting of PHA-680632, AKI-001, VX-680, PHA-739358, MLN8054, MLN 8237, C1368 and siRNA which hybridize selectively to Aurora kinase and said EGFR inhibitor is selected from the group consisting of erlotinib, cetuximab, gefinitib, and panitumumab.

16. The pharmaceutical composition of claim 15, further comprising at least one anti-proliferative agent.

17. The pharmaceutical composition of claim 16, wherein said at least one anti-proliferative agent is selected from the group consisting of a toxin, saporin, ricin, abrin, ethidium bromide, diptheria toxin, Pseudomonas exotoxin, an alkylating agent, a nitrogen mustards, chlorambucil, cyclophosphamide, isofamide, mechlorethamine, melphalan, uracil mustard; aziridines, thiotepa; a methanesulphonate ester, busulfan; carmustine, lomustine, streptozocin; cisplatin, carboplatin; mitomycin, procarbazine, dacarbazine and altretamine, bleomycin, amsacrine, dactinomycin, daunorubicin, idarubicin, mitoxantrone, doxorubicin, etoposide, teniposide, plicamydin, methotrexate, trimetrexate; fluorouracil, fluorodeoxyuridine, CB3717, azacitidine, cytarabine, floxuridine; mercaptopurine, 6-thioguanine, fludarabine, pentostatin; asparginase, hydroxyurea, vincristine, vinblastine, paclitaxel (Taxol), estrogens; conjugated estrogens; ethinyl estradiol; diethylstilbesterol; chlortrianisen; idenestrol; hydroxyprogesterone caproate, medroxyprogesterone, megestrol; testosterone, testosterone propionate, fluoxymesterone, methyltestosterone, abarelix abiraterone acetate, Degarelix, prednisone, dexamethasone, methylprednisolone, and prednisolone, leuprolide acetate, goserelin acetate, tamoxifen, flutamide, mitotane, and aminoglutethimide, combretastatin and derivatives thereof, bevacizumab (Avastin®), sorafenib, trastuzumab, Raf265 and temsirolimus.

18. A kit comprising compositions useful for the treatment of cancer, comprising the pharmaceutical compositions of claim 15.


SEQUENCE LISTING (2 sequences)

SEQ ID NO: 1 - DNA, 21 bp, Artificial Sequence
  Synthetic sequence
tcccagcgcattcctttgcaa

SEQ ID NO: 2 - DNA, 21 bp, Artificial Sequence
  Synthetic sequence
caccttcggcatcctaatatt